(19) United States
(12) Patent Application Publication
(10) Pub. No.: US 2005/0031610 A1
(43) Pub. Date: Feb. 10, 2005

(54) CD44-related fragments, compositions and methods

(76) Inventors: Tae-Wan Kim, Old Tappan, NJ (US); Kwang Mook Jung, Aliso Viejo, CA (US)

Correspondence Address: Cooper & Dunham LLP, 1185 Avenue of the Americas, New York, NY, 10036, US

(21) Appl. No.: 10/623,442

(22) Filed: Jul. 18, 2003

Related U.S. Application Data
(60) Provisional application No. 60/397,077, filed on Jul. 19, 2002.

(51) Int. Cl. G01N 33/53; G01N 33/567; A61K 39/395; A61K 39/00; C07K 14/74; C07K 16/28

(52) U.S. Cl. 424/144.1; 424/185.1; 530/350; 530/388.22; 435/7.2

(57) ABSTRACT

Described is an isolated CD44 fragment, which fragment comprises the amino acid sequence of a fragment formed in a CD44⁺ cell in the presence of extracellular hyaluronan and of intracellular γ-secretase and metalloprotease. Also described are related polypeptides, pharmaceutical compositions, antibodies, assay methods, quantitation methods, therapeutic methods and articles of manufacture.

This application claims priority of provisional application U.S. Ser. No. 60/397,077, filed Jul. 19, 2002, the contents of which are incorporated herein by reference.

The invention described herein was made with government support under NIH Grant R01-AG18026. Accordingly, the United States government has certain rights in this invention.

Throughout this application, various references are cited. Disclosure of these references in their entirety is hereby incorporated by reference into this application to more fully describe the state of the art to which this invention pertains.

### BACKGROUND OF THE INVENTION

γ-Secretase is an unusual protease that cleaves amyloid precursor protein, Notch, and ErbB4 within the transmembrane region. CD44 is a broadly distributed cell surface adhesion receptor implicated in various cellular processes, including neuronal development, cell migration, tumor growth and metastasis. Among many reported CD44-associated biological processes, the binding of CD44 to high molecular weight forms of hyaluronate (HA), an extracellular matrix component, has been reported to induce cell growth arrest.

### SUMMARY OF THE INVENTION

This invention provides an isolated CD44 fragment, which fragment comprises the amino acid sequence of a fragment formed in a CD44⁺ cell in the presence of extracellular hyaluronan and of intracellular γ-secretase and metalloprotease.

This invention further provides a composition comprising the instant CD44 fragment and a pharmaceutically acceptable carrier.

This invention further provides an antibody which specifically binds to the instant CD44 fragment.

This invention further provides two methods for determining whether an agent increases the amount of CD44 fragment formed in a CD44⁺ cell. The first method comprises the steps of (a) contacting the CD44⁺ cell with the agent, (b) determining the amount of γ-secretase-generated CD44 fragment present in the CD44⁺ cell, and (c) comparing the amount of CD44 fragment determined in step (b) with the amount of CD44 fragment present in a CD44⁺ cell not contacted with the agent, a greater amount of CD44 fragment in the cell contacted with the agent indicating that the agent increases the amount of CD44 fragment formed.

The second method comprises the steps of (a) contacting the agent with a CD44⁺ membrane fragment in the presence of hyaluronan, γ-secretase and metalloprotease, (b) determining the amount of CD44 fragment formed in step (a), and (c) comparing the amount of CD44 fragment determined in step (b) with the amount of CD44 fragment formed in the absence of the agent, a greater amount of CD44 fragment formed in the presence of the agent indicating that the agent increases the amount of CD44 fragment formed.

This invention also provides a method for increasing the amount of CD44 fragment formed in a CD44⁺ cell, which method comprises introducing into the cell γ-secretase and/or a γ-secretase agonist.

This invention further provides a method for determining the amount of CD44 fragment in a sample, which comprises contacting the sample with an antibody which specifically binds to the instant CD44 fragment under conditions permitting the formation of a complex between the antibody and the CD44 fragment, and determining the amount of complex so formed, thereby determining the amount of CD44 fragment in the sample.

This invention also provides two methods for treating a subject afflicted with a CD44-associated disorder. The first method comprises administering to the subject a therapeutically effective amount of γ-secretase or a γ-secretase agonist, thereby treating the subject. The second method comprises administering to the subject a therapeutically effective amount of the instant CD44 fragment, thereby treating the subject.

Finally, this invention provides an article of manufacture comprising a packaging material having therein the instant CD44 fragment, and a label indicating a use for the CD44 fragment in treating a CD44-associated disorder.

### BRIEF DESCRIPTION OF THE FIGURES

FIG. 1. The accumulation of membrane-associated C-terminal derivatives of CD44 by a γ-secretase inhibitor. The CHO cells transfected with constructs encoding CD44 with C-terminal V5-tag were incubated in the presence of the indicated concentrations of γ-secretase inhibitor Compound E (Cpd. E) for 16 hours. Detergent lysates were analyzed by Western blot analyses (4-20% Tris-Glycine SDS-PAGE) using anti-V5 antibody.

FIG. 2. CD44 C-terminal fragments accumulate in fibroblasts lacking both PS1 and PS2. Immortalized embryonic fibroblasts derived from wild-type or PS1−/− PS2−/− double knock-out mice (DKO) were transfected with full-length CD44 constructs, lysed, and subjected to Western blot analyses using anti-V5 antibody (A), anti-PS1loop or anti-PS2loop antibodies (B).

FIG. 3. CD44 C-terminal fragments accumulate in HEK293 cells co-expressing dominant-negative forms of PS1 and PS2. Parental HEK293 cells (293) and stable HEK293 cells co-expressing wild-type PS1 and PS2 (dW) or dominant negative forms of PS1 and PS2 (dAsp:D385A-PS1 and dAsp:D366A-PS2, respectively). Cell lysates were subjected to Western blot analyses using the indicated antibodies.

FIG. 4. In vitro generation of the CD44 intracellular domain (CD44-ICD). Membrane fractions prepared from either dW or dAsp cells transfected with full-length CD44 were incubated at 37° C. for the indicated hours with or without Cpd E (1 mM) or MG132 (50 mM). After the incubation, both the pellet (A) and the supernatant (B) were analyzed by Western blot analyses by Western blotting using anti-V5 antibody.

FIG. 5. (A) Schematic representations of truncated CD44 constructs and putative cleavage sites of CD44. Starting amino acid sequences of truncated (ΔE) or intracellular (ICD) CD44 constructs and predicated proteolytic cleavage sites are indicated by arrows. Full-length (FL) and all ICD constructs contain C-terminal V5 tag, whereas all ΔE constructs harbor N-terminal HA tag located immediately after the signal peptide in addition to the C-terminal V5 tag. (B) Expression of truncated CD44 constructs in HEK293 cells. The lysates from the cells transfected with indicated constructs were analyzed by Western blot analyses. The locations of precursor polypeptide encoded by each constructs as well as the cleavage products (e.g. S1, T1, and T2) are shown on the right side of each blot. The lysates from full-length CD44-transfected cells grown in the absence (FL) or presence (FL+hyaluronan) of hyaluronan are shown as controls. The treatment with hyaluronan caused the increased generation of S1 and T2 cleavage (FL vs. FL+hyaluronan).

FIG. 6. Ligand-induced generation of CD44-ICD. Cells were treated for 24 hours with solvent only (Control), hyaluronan, hyaluronan plus Cpd. E, phorbol ester TPA, and TPA plus hyaluronan, and the lysates were analyzed by Western blotting using anti-V5 antibody. Note that only T2 cleavage products were selectively affected by the treatment with Cpd. E, but not C1 cleavage.

FIG. 7. Effects of a γ-secretase inhibitor in hyaluronan-mediated growth retardation. HEK293 cells were transfected with constructs encoding both full-length CD44 and enhance green fluorescence protein (EGFP) in a single plasmid. Identical number of transfected cells were plated into multiple dishes and incubated with hyaluronan, hyaluronan plus Cpd. E, TPA, and TPA plus Cpd. E. After 40 hour incubation, the number of EGFP-positive cells were counted. Values represent the mean±s.d.

### DETAILED DESCRIPTION OF THE INVENTION

Definitions

As used in this application, except as otherwise expressly provided herein, each of the following terms shall have the meaning set forth below.

“Administering” shall mean delivering in a manner which is effected or performed using any of the various methods and delivery systems known to those skilled in the art. Administering can be performed, for example, topically, intravenously, pericardially, orally, via implant, transmucosally, transdermally, intramuscularly, subcutaneously, intraperitoneally, intrathecally, intralymphatically, intralesionally, or epidurally. Administering can also be performed, for example, once, a plurality of times, and/or over one or more extended periods.

“Agent” shall mean any chemical entity, including, without limitation, a glycomer, a protein, an antibody, a lectin, a nucleic acid, a small molecule, and any combination thereof.

“Amino acid,” “amino acid residue” and “residue” are used interchangeably herein to refer to an amino acid that is incorporated into a protein, polypeptide or peptide. The amino acid can be a hydrophobic, hydrophilic, charged, and/or uncharged amino acid, a naturally occurring amino acid or a derivative of an amino acid that can function in a similar manner as the naturally occurring amino acid.

“Amino acid derivative” means an amino acid other than one of the 20 amino acids commonly referred to as the 20 naturally occurring amino acids. Many amino acids, including the terminal amino acids, may be modified by natural processes, such as processing and other post-translational modifications, or by chemical modification techniques well known in the art. Common modifications that occur naturally are described in basic texts, detailed monographs, and the research literature, and they are well known to those of skill in the art.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol. 182: 626-646 (1990)) and Rattan et al. (Ann. N.Y. Acad. Sci. 663:48-62 (1992)).

“Antibody” includes, by way of example, both naturally occurring antibodies (e.g., IgG, IgM, IgE and IgA) and non-naturally occurring antibodies. The term “antibody” also includes polyclonal and monoclonal antibodies, and fragments thereof (e.g., antigen-binding portions). Furthermore, the term “antibody” includes chimeric antibodies, wholly synthetic antibodies, human and humanized antibodies, and fragments thereof.

“CD44 fragment” shall mean a free polypeptide which constitutes a portion of the membrane-bound protein CD44. In the preferred embodiment, the CD44 fragment constitutes a portion of the cytoplasmic domain of CD44.

“CD44⁺ cell” shall mean a cell comprising the membrane-bound protein CD44. In one embodiment, the CD44⁺ cell is a human CD44⁺ cell.

“CD44-associated disorder” shall include, without limitation, (a) a disorder characterized by a reduced level of CD44 fragment in the CD44⁺ cells of an afflicted subject, and (b) a disorder ameliorated by an increase in the level of CD44 fragment in the CD44⁺ cells of an afflicted subject.

“Mammalian cells” include, without limitation, normal, abnormal and transformed mammalian cells, and are exemplified by neurons, epithelial cells, muscle cells, blood cells, immune cells, stem cells, osteocytes, endothelial cells and blast cells.

“Pharmaceutically acceptable carriers” are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer=s dextrose, dextrose and sodium chloride, lactated Ringer=s and fixed oils. Intravenous vehicles include fluid and nutrient replenishers, electrolyte replenishers such as those based on Ringer=s dextrose, and the like. Preservatives and other additives may also be present, such as, for example, antimicrobials, antioxidants, chelating agents, inert gases, and the like.

“Polypeptide” means a polymer of amino acid residues. The amino acid residues can be naturally occurring or chemical analogues thereof. Polypeptides, peptides and proteins can also include modifications such as glycosylation, lipid attachment, sulfation, hydroxylation, and ADP-ribosylation.

“Sample”, when used in connection with the instant methods, includes, but is not limited to, any body tissue, skin lesion, blood, serum, plasma, cerebrospinal fluid, lymphocyte, urine, exudate, or supernatant from a cell culture.

“Specifically bind” shall mean that, with respect to the binding of an antibody to its antigen, the antibody binds to the antigen with a greater affinity than that with which it binds to most other antigens. In the preferred embodiment, the antibody binds to the antigen with a greater affinity than that with which it binds to all other antigens.

“Subject” shall mean any animal, such as a mammal or a bird, including, without limitation, a cow, a horse, a sheep, a pig, a dog, a cat, a rodent such as a mouse or rat, a chicken and a primate. In the preferred embodiment, the subject is a human.

“Therapeutically effective amount” means an amount sufficient to treat a subject. A person of ordinary skill in the art can perform simple titration experiments to determine such amount.

“Treating” means either slowing, stopping or reversing the progression of a disorder. As used herein, “treating” also means the amelioration of symptoms associated with the disorder.

Embodiments of the Invention

This invention provides an isolated CD44 fragment, which fragment comprises the amino acid sequence of a fragment formed in a CD44⁺ cell in the presence of extracellular hyaluronan and of intracellular γ-secretase and metalloprotease. In a preferred embodiment, the fragment formed in the CD44⁺ cell is a cleavage product of γ-secretase.

This invention also provides a polypeptide comprising the instant CD44 fragment, wherein at least one amino acid residue thereof is chemically modified. In one embodiment, the polypeptide contains at least one amino acid residue which is an amino acid derivative.

This invention further provides a composition comprising the instant CD44 fragment and a pharmaceutically acceptable carrier. In a preferred embodiment, the carrier is a 16 amino acid polypeptide of the Antennapedia protein of the Drosophila fruit fly, said polypeptide currently known as PENETRATIN®.

This invention also provides an antibody which specifically binds to the instant CD44 fragment. In one embodiment, the antibody is labeled with a detectable moiety. In another embodiment, the detectible moiety is a radioisotope, an enzyme, a fluorogenic material, a chemiluminescent material or an electrochemical material.

This invention further provides two methods for determining whether an agent increases the amount of CD44 fragment formed in a CD44⁺ cell. The first method comprises the steps of (a) contacting the CD44⁺ cell with the agent, (b) determining the amount of γ-secretase-generated CD44 fragment present in the CD44⁺ cell, and (c) comparing the amount of CD44 fragment determined in step (b) with the amount of CD44 fragment present in a CD44⁺ cell not contacted with the agent, a greater amount of CD44 fragment in the cell contacted with the agent indicating that the agent increases the amount of CD44 fragment formed.

The second method comprises the steps of (a) contacting the agent with a CD44⁺ membrane fragment in the presence of hyaluronan, γ-secretase and metalloprotease, (b) determining the amount of CD44 fragment formed in step (a), and (c) comparing the amount of CD44 fragment determined in step (b) with the amount of CD44 fragment formed in the absence of the agent, a greater amount of CD44 fragment formed in the presence of the agent indicating that the agent increases the amount of CD44 fragment formed.

This invention also provides a method for increasing the amount of CD44 fragment formed in a CD44⁺ cell, which method comprises introducing into the cell γ-secretase and/or a γ-secretase agonist. In one embodiment, the CD44⁺ cell is a mammalian cell. In another embodiment, the CD44⁺ cell is a human cell.

This invention also provides a method for determining the amount of CD44 fragment in a sample, which method comprises contacting the sample with an antibody which specifically binds to the instant CD44 fragment under conditions permitting the formation of a complex between the antibody and the CD44 fragment, and determining the amount of complex so formed, thereby determining the amount of CD44 fragment in the sample. In a preferred embodiment, the sample comprises a CD44⁺ cell. In another embodiment, the CD44⁺ cell is a mammalian cell. In a further embodiment, the CD44⁺ cell is a human cell.

This invention also provides two methods for treating a subject afflicted with a CD44-associated disorder. The first method comprises administering to the subject a therapeutically effective amount of γ-secretase or a γ-secretase agonist, thereby treating the subject. The second method comprises administering to the subject a therapeutically effective amount of the instant CD44 fragment, thereby treating the subject. In one embodiment, the CD44-associated disorder is cancer. In another embodiment, the CD44-associated disorder is streptococcal invasion.

Finally, this invention provides an article of manufacture comprising a packaging material having therein the instant CD44 fragment, and a label indicating a use for the CD44 fragment in treating a CD44-associated disorder. In one embodiment, the CD44-associated disorder is cancer. In another embodiment, the CD44-associated disorder is streptococcal invasion.

This invention will be better understood from the Experimental Details that follow. However, one skilled in the art will readily appreciate that the specific methods and results discussed are merely illustrative of the invention as described more fully in the claims which follow thereafter.

### Experimental Details

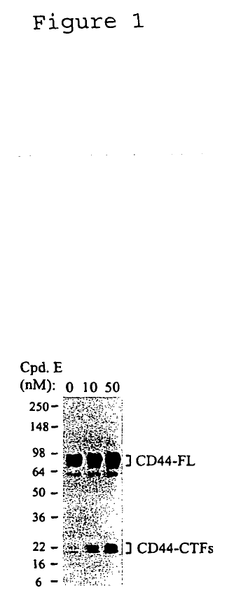

When constructs encoding full-length CD44 with C-terminal V5 epitope tag were transiently expressed, full-length CD44 (˜85 kDa) was constitutively processed to generate membrane-associated C-terminal fragments with apparent molecular weights of ˜19 to 22 kDa (FIG. 1). First, experiments were performed to determine whether a γ-secretase inhibitor blocks the putative intramembrane cleavage of CD44 to cause an accumulation of CD44 similar to that observed for APP and ErbB4 (Lee, H.-J., Jung, K.-M., Huang, Y. Z., Bennett, L. B., Lee, J. S., Mei, L. and Kim, T.-W., Presenilin-dependent γ-secretase-like intramembrane cleavage of ErbB4. J. Biol. Chem., 277:6318-6329 (2002)). Treatment of the CD44-transfected cells with the potent γ-secretase inhibitor Compound E (Seiffert, D., et al. J. Biol. Chem. 275, 34086-34091, 2000) led to the accumulation of transgene-derived CD44 C-terminal fragments in CHO cells (FIG. 1).

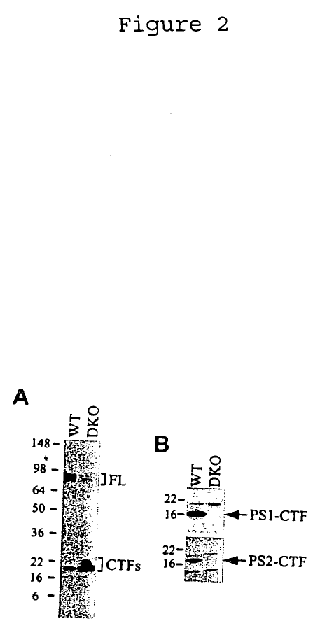

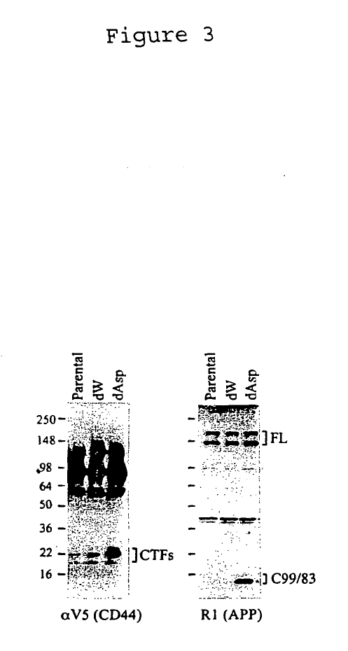

Next, the effects of the deficiency in presenilin-dependent γ-secretase activity was studied. For this purpose, the accumulation of the membrane-associated CD44 C-terminal fragments was examined in either fibroblasts lacking both presenilin 1 (PS1) and presenilin 2 (PS2) (FIG. 2) or stable 293 cells harboring constructs encoding dominant-negative mutant (dAsp:D385A-PS1 and dAsp:D366A-PS2) forms of PS1 and PS2. The accumulation of the membrane-associated CD44 C-terminal fragments was dramatically elevated in both PS1 and PS2 double knock-out cells (FIG. 2) and dAsp cells (FIG. 3), as compared to wild-type counter parts (WT and dW, respectively).

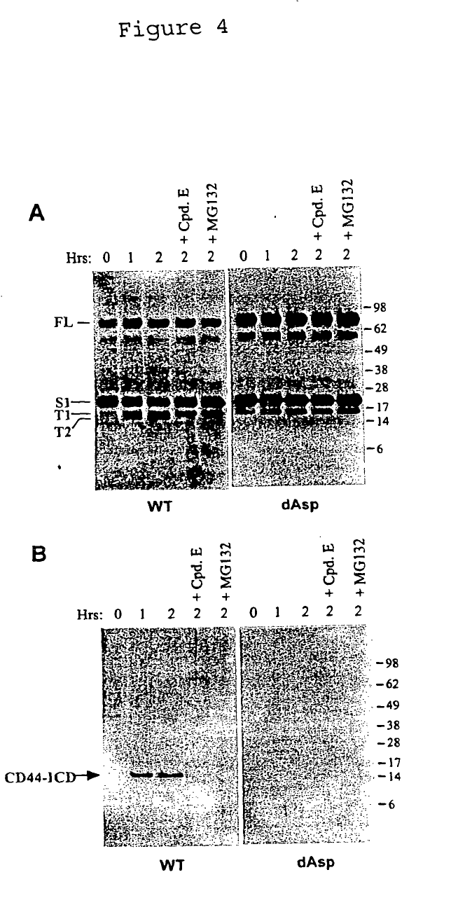

To directly demonstrate the intramembrane cleavage of CD44 and the subsequent release of soluble intracellular domain of CD44 (CD44-ICD), in vitro CD44-ICD generation experiments were performed using the membrane fraction prepared from either wild-type presenilin-expressing 293 cells (WT) or dominant-negative presenilin-expressing 293 cells (dasp) (FIG. 4). The incubation of the cytosol-free membrane fractions from dW cells transfected with CD44 led to the generation of two fragments (T1 and T2) in the membrane fractions (FIG. 4A) and the release of V5-epitope-tagged CD44-ICD into the soluble fraction (FIG. 4B). Interestingly, the generation of T1 fragments were not inhibited in both dW and dasp cells and were also not blocked by the treatment with Compound E, indicating that T1 cleavage is presenilin-independent (FIG. 4). The release of CD44-ICD was increased in a time-dependent manner in dW cells, whereas inhibition occurred in both membrane fractions from dasp cells and dW cells incubated with Compound E or MG132 (FIG. 4B).

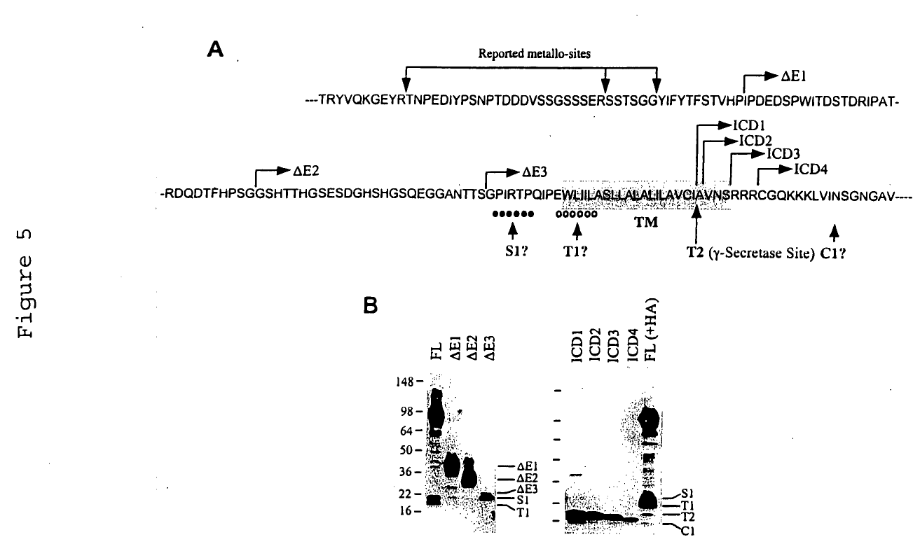

To map the cleavage sites for S1, T1 and T2 cleavage, constructs encoding either the truncated CD44 lacking the majority of the extracellular regions (ΔE1, ΔE2, and ΔE3) or the intracellular domains (ICD1, ICD2, ICD3, and ICD4) of CD44 were expressed (FIG. 5). The location of S1, T1, T2, and predicted cytoplasmic cleavage (C1) were compared in Western blots of lysates from the cells transfected with the indicated constructs (FIG. 5B). The results indicate that S1 cleavage occurs at regions that are different from previously reported metalloprotease sites (Kajita et al. J. Cell. Biol. 153, 893-904, 2001). T1 cleavage is predicted to occur either within the tramsmembrane region or near the junction between the extracellular domain and the transmembrane region.

To study the biological role of γ-secretase-mediated cleavage of CD44, the effects of inhibition of CD44 intramembrane cleavage in CD44-associated biological processes were tested.

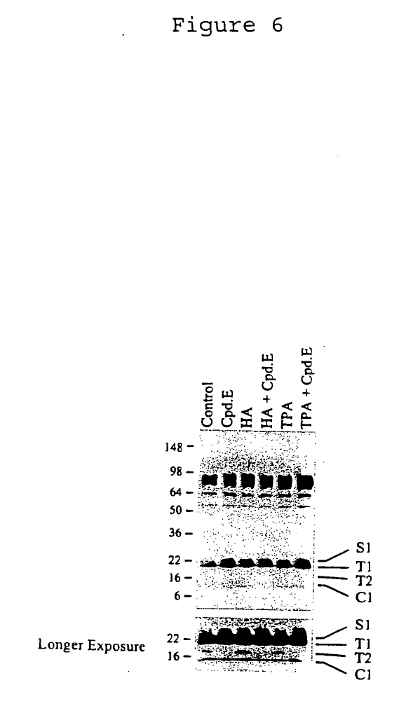

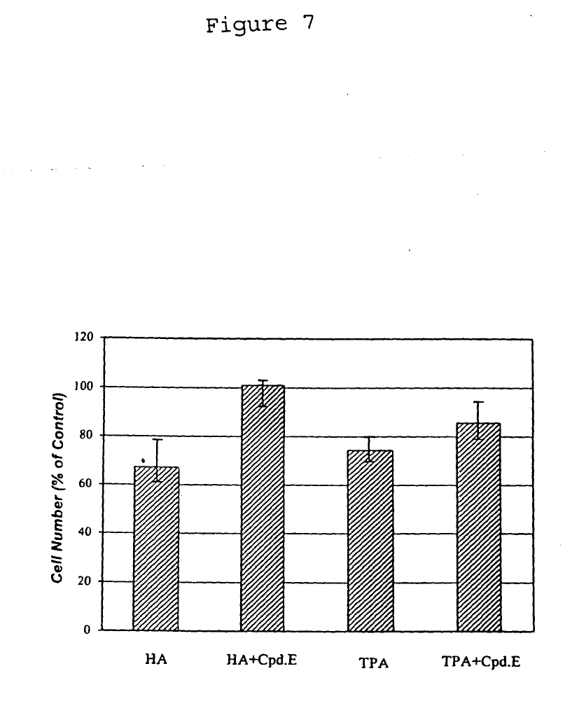

It has been previously reported that high molecular weight forms of hyaluronan (HA) induce cell growth arrest (Morrison et al., Genes and Development, 15, 968-980, 2001; Herrlich et al., Ann. NY Acad. Sci. 910, 106-118, 2000). The treatment of cells with the high molecular weight forms of HA led to the generation of CD44-ICD in a γ-secretase-dependent manner (FIG. 6A). The levels of S1 and T1 cleavage products were also elevated, although γ-secretase inhibitor Compound E didn't inhibit the accumulation of these fragments. HA treatment induced growth inhibition in the cells transfected with CD44 (FIG. 7). However, treatment with Compound E reverted the growth-inhibition induced by HA, indicating that γ-secretase-mediated cleavage of CD44 is associated with the observed growth inhibition induced by HA. The phorbol-ester TPA showed similar effects to a lesser extent.

Discussion

γ-Secretase is an unusual protease that cleaves amyloid precursor protein, Notch, and ErbB4 within the transmembrane region. This study demonstrates that CD44 is a novel substrate for presenilin-dependent γ-secretase. CD44 is a broadly distributed cell surface adhesion receptor implicated in various cellular processes, including neuronal development, cell migration, tumor growth and metastasis. Among many reported CD44-associated biological processes, the binding of CD44 to high molecular weight forms of hyaluronate (HA), an extracellular matrix component, has been reported to induce cell growth arrest. This study shows that HA- or phorbol ester-induced ectodomain shedding of full-length CD44 produces a ˜25 kDa membrane-associated C-terminal fragment (CD44-CTF). In the cells devoid of functional presenilins (e.g. cells co-expressing dominant negative forms of PS1 and PS2 or double knock-out cells), subsequent intramembrane cleavage of CD44-CTF was inhibited, leading to enhanced accumulation of CD44-CTF. In addition, CD44-CTF levels were also increased by treatment of cells with a synthetic γ-secretase inhibitor. The presenilin-dependent release of the intracellular domain of CD44 (CD44-ICD) was also detected using an in vitro γ-secretase assay. To evaluate the biological significance of presenilin-dependent CD44 intramembrane cleavage, the HA-induced growth regulation mediated by CD44 was examined. In CD44-expressing cells, the HA treatment led to an inhibition of cell proliferation that was reverted in the presence of the γ-secretase inhibitor. These studies indicate that CD44 undergoes presenilin-dependent intramembrane proteolysis that may be critical for regulating cell growth and other CD44-mediated cellular processes.

References

Creighton, T. E., Proteins—Structure and Molecular Properties, 2nd Ed., W.H. Freeman and Company, New York (1993).
Herrlich, et al., Ann. NY Acad. Sci. 910:106-118 (2000).
Kajita, et al., J. Cell. Biol. 153:893-904 (2001).
Lee, H.-J., Jung, K.-M., Huang, Y. Z., Bennett, L. B., Lee, J. S., Mei, L. and Kim, T.-W., Presenilin-dependent γ-secretase-like intramembrane cleavage of ErbB4. J. Biol. Chem. 277:6318-6329 (2002).
Morrison, et al., Genes and Development 15:968-980 (2001).
Rattan, et al., Ann. N.Y. Acad. Sci. 663:48-62 (1992).
Seiffert, D., et al., J. Biol. Chem. 275:34086-34091 (2000).
Seifter, et al., Meth. Enzymol. 182:626-646 (1990).
Wold, F., Posttranslational Covalent Modification of Proteins, B. C. Johnson, Ed., Academic Press, New York, pp. 1-12 (1983).

1. An isolated CD44 fragment, which fragment comprises the amino acid sequence of a fragment formed in a CD44⁺ cell in the presence of extracellular hyaluronan and of intracellular γ-secretase and metalloprotease.

2. The fragment of claim 1, wherein the fragment formed in the CD44⁺ cell is a cleavage product of γ-secretase.

3. A polypeptide comprising the CD44 fragment of claim 1, wherein at least one amino acid residue thereof is an amino acid derivative.

4. A composition comprising the CD44 fragment of claim 1 and a pharmaceutically acceptable carrier.

5. The composition of claim 4, wherein the carrier is a 16 amino acid polypeptide of the Antennapedia protein of the Drosophila fruit fly.

6. An antibody which specifically binds to the CD44 fragment of claim 1.

7. The antibody of claim 6, wherein the antibody is labeled with a detectable moiety.

8. The antibody of claim 7, wherein the detectible moiety is a radioisotope, an enzyme, a fluorogenic material, a chemiluminescent material or an electrochemical material.

9. A method for determining whether an agent increases the amount of CD44 fragment formed in a CD44⁺ cell, which comprises the steps of
(a) contacting the CD44⁺ cell with the agent;
(b) determining the amount of γ-secretase-generated CD44 fragment present in the CD44⁺ cell; and
(c) comparing the amount of CD44 fragment determined in step (b) with the amount of CD44 fragment present in a CD44⁺ cell not contacted with the agent, a greater amount of CD44 fragment in the cell contacted with the agent indicating that the agent increases the amount of CD44 fragment formed.

10. A method for determining whether an agent increases the amount of CD44 fragment formed in a CD44⁺ cell, which comprises the steps of
(a) contacting the agent with a CD44⁺ membrane fragment in the presence of hyaluronan, γ-secretase and metalloproteinase;
(b) determining the amount of CD44 fragment formed in step (a); and
(c) comparing the amount of CD44 fragment determined in step (b) with the amount of CD44 fragment formed in the absence of the agent, a greater amount of CD44 fragment formed in the presence of the agent indicating that the agent increases the amount of CD44 fragment formed.

11. A method for increasing the amount of CD44 fragment formed in a CD44⁺ cell, which method comprises introducing into the cell γ-secretase and/or a γ-secretase agonist.

12. A method for determining the amount of CD44 fragment in a sample, which method comprises contacting the sample with the antibody of claim 6 under conditions permitting the formation of a complex between the antibody and the CD44 fragment, and determining the amount of complex so formed, thereby determining the amount of CD44 fragment in the sample.

13. The method of claim 12, wherein the sample comprises a CD44⁺ cell.

14. The method of claim 11 or 13, wherein the cell is a mammalian cell.

15. The method of claim 14, wherein the cell is a human cell.

16. A method for treating a subject afflicted with a CD44-associated disorder comprising administering to the subject a therapeutically effective amount of γ-secretase or a γ-secretase agonist, thereby treating the subject.

17. A method for treating a subject afflicted with a CD44-associated disorder comprising administering to the subject a therapeutically effective amount of the CD44 fragment of claim 1, thereby treating the subject.

18. The method of claim 16 or 17, wherein the CD44-associated disorder is cancer.

19. The method of claims 16 or 17, wherein the CD44-associated disorder is streptococcal invasion.

20. An article of manufacture comprising a packaging material having therein the CD44 fragment of claim 1, and a label indicating a use for the CD44 fragment in treating a CD44-associated disorder.

21. The article of claim 20, wherein the CD44-associated disorder is cancer.

22. The article of claim 20, wherein the CD44-associated disorder is streptococcal invasion.


SEQUENCE LISTING (1 sequence)

SEQ ID NO: 1 - protein, 158 aa, artificial
  Truncated CD44 construct and putative cleavage sites of CD44
TRYVQKGEYRTNPEDIYPSNPTDDDVSSGSSSERSSTSGGYIFYTFSTVHPIPDEDSPWITDSTDRIPATRDQDTFHPSGGSHTTHGSESDGHSHGSQEGGANTTSGPIRTPQIPEWLIILASLLALALILAVCIAVNSRRRCGQKKKLVINSGNGAV